(19) United States
(12) Patent Application Publication
(10) Pub. No.: US 2021/0149101 A1
(43) Pub. Date: May 20, 2021

(54) Multicore Fiber Instrument with 3D-Printed Distal Optics

(71) Applicant: Eric Swanson, Gloucester, MA (US)

(72) Inventor: Eric Swanson, Gloucester, MA (US)

(21) Appl. No.: 16/864,056

(22) Filed: Apr. 30, 2020

Related U.S. Application Data
(60) Provisional application No. 62/946,624, filed on Dec. 11, 2019.
(60) Provisional application No. 62/935,444, filed on Nov. 14, 2019.

(51) Int. Cl. F21V 8/00; G02B 23/24; G02B 23/26; G02B 6/02

(52) U.S. Cl. CPC G02B 6/0008; G02B 23/2469; A61B 1/07; G02B 6/02042; G02B 23/26

(57) ABSTRACT

A multicore fiber light transfer system includes a multicore fiber having a proximal end and a distal end and at least three optical cores. The multicore fiber transferring light from the proximal end to the distal end and collecting light from a target at the distal end and transferring the collected light to the proximal end. Distal optics is 3D printed near the distal end of the multicore fiber. The distal optics includes a first element having a surface that is aligned to one core of the multicore fiber with a first shape such that the first element projects the light transferred from the proximal end in a first desired direction with a first desired beam shape and having a second element comprising a surface that is aligned to another core of the multicore fiber with a second shape such that the second element collects light from a desired location on the target.

### CROSS REFERENCE TO RELATED APPLICATION

The present application is a non-provisional application of U.S. Provisional Patent Application No. 62/935,444, filed on Nov. 14, 2019, entitled “Multicore Fiber with 3D Printed Distal Optics” and is a non-provisional application of U.S. Provisional Patent Application No. 62/946,624, filed on Dec. 11, 2019, also entitled “Multicore Fiber with 3D Printed Distal Optics”. The entire contents of U.S. Provisional Patent Application Nos. 62/935,444 and 62/946,624 are herein incorporated by reference.

### INTRODUCTION

The present teaching relates to medical and non-medical applications for delivering and/or collecting light, and/or performing sensing, and/or performing optical imaging, and/or performing optical therapy of a sample at the distal end of an optical waveguide. There are many medical and non-medical needs for performing optical imaging or sensing of a sample (e.g. human organ or sample in hard to reach places). In some applications that rely on the delivering and/or collecting of light, the range and/or optical properties of a sample or target are determined. Optical properties can include, for example, absorption, reflection, refractive index, birefringence, dispersion, scattering, spectral characteristics, fluorescence, and other properties. The optical properties can be determined as a function of wavelength. In addition, the optical properties can be determined at a point, in a small volume, and/or can be spatially or spectrally resolved along one dimension, or multiple dimensions. In addition, the distance or range to a sample or target can be determined.

Single-mode optical fibers are inexpensive and flexible and commonly used to transmit light along a fiber-based optical instrument. But single-mode fiber by itself has limited capabilities. For example, to perform imaging using a single-mode fiber usually requires scanning of the light emitted and/or collected from the single-mode fiber. These known techniques suffer from a variety of significant limitations such as: the endoscopic probe being too thick and/or not flexible enough to access important regions within the human body; an inability to fit inside existing ports of clinical and non-clinical instruments; the endoscope or the system it attaches being too expensive; the endoscope being less reliable than desired; and/or the scanning mechanism introducing optical image artifacts, such as non-uniform rotation distortion. A significant advance over these limitations in prior art fiber-based instruments is needed to open up new clinical and non-clinical applications and to perform better in existing ones.

### BRIEF DESCRIPTION OF THE DRAWINGS

The present teaching, in accordance with preferred and exemplary embodiments, together with further advantages thereof, is more particularly described in the following detailed description, taken in conjunction with the accompanying drawings. The person skilled in the art will understand that the drawings, described below, are for illustration purposes only. The drawings are not necessarily to scale, emphasis instead generally being placed upon illustrating principles of the teaching. The drawings are not intended to limit the scope of the Applicant's teaching in any way. Also note for simplicity, some of the drawings show dimensions and/or beam propagation (e.g. beam divergence) that is not to scale or proportion or exact location with respect to the target or sample.

FIG. 1A illustrates a known general system concept of an endoscopic instrument using a multicore optical fiber with at least three optical cores.

FIG. 1B illustrates an end-view cross-section of the multicore optical fiber of FIG. 1A.

FIG. 2 illustrates examples of known 3D printed optics that can be realized.

FIG. 3 illustrates an embodiment of a multicore fiber light transfer system in side-view cross section with 3D-printed distal optics fold mirrors of the present teaching.

FIG. 4 illustrates an embodiment of a multicore fiber light transfer system in side-view cross section with 3D-printed distal optics fold mirrors with additional distal structure of the present teaching.

FIG. 5 illustrates an embodiment of a multicore fiber light transfer system in side-view cross section with 3D-printed distal optics comprising a lens before fold mirrors of the present teaching.

FIG. 6 illustrates an embodiment of a multicore fiber light transfer system in side-view cross section with 3D-printed distal optics comprising fold mirrors positioned forward of lenses of the present teaching.

### DESCRIPTION OF VARIOUS EMBODIMENTS

The present teaching will now be described in more detail with reference to exemplary embodiments thereof as shown in the accompanying drawings. While the present teaching is described in conjunction with various embodiments and examples, it is not intended that the present teaching be limited to such embodiments. On the contrary, the present teaching encompasses various alternatives, modifications and equivalents, as will be appreciated by those of skill in the art. Those of ordinary skill in the art having access to the teaching herein will recognize additional implementations, modifications, and embodiments, as well as other fields of use, which are within the scope of the present disclosure as described herein.

Reference in the specification to “one embodiment” or “an embodiment” means that a particular feature, structure, or characteristic described in connection with the embodiment is included in at least one embodiment of the teaching. The appearances of the phrase “in one embodiment” in various places in the specification are not necessarily all referring to the same embodiment.

It should be understood that the individual steps of the methods of the present teaching can be performed in any order and/or simultaneously as long as the teaching remains operable. Furthermore, it should be understood that the apparatus and methods of the present teaching can include any number or all of the described embodiments as long as the teaching remains operable.

It should be understood that the word “fiber” and the word “core” are used throughout the specification in a somewhat interchangeable manner. In particular, it should be understood by those of skill in the art that when multiple cores are described as embedded in a common cladding, there is an equivalent embodiment with multiple optical fibers, each with a core and a cladding embedded in a second outer common cladding. Such cores could be single-mode, few-mode, and/or multi-mode optical cores.

The present teaching relates to the many medical and non-medical applications for delivering and/or collecting light and/or performing optical imaging of a sample in hard to reach places. In this disclosure, the word “light” is intended to be a general term for any radiation, for example, in the wavelength range from ultraviolet to infrared, including the entire visible spectrum. Also, it should be understood that the terms “waveguide” and “fiber” are used interchangeably in this disclosure as an optical fiber is a type of waveguide. It should also be understood that the term “endoscope” as used herein is intended to have a broad meaning to include medical devices such as catheters, guidewires, laparoscopes, trocars, borescopes, needles, and various minimally invasive and robotic surgical devices. In addition, the present teaching is not limited to use in endoscopes but, in fact, has a wide variety uses in fiber-based instruments that are housed in numerous types of packages and apply to a variety of illumination and/or measurement, ranging, and sensing applications.

The use of multicore (or multimode) optical fiber according to the present teaching, instead of single-mode optical fiber in a fiber-based optical instrument, offers dramatic advantages for applications. This is because, for example, multicore fibers can be used such that each core, or group of cores, supports a different aspect of the measurement. For example, some core or cores could be used to transmit light to illuminate a sample and other core or cores could be used to transmit light collected from a sample, and some core or cores could be used for both purposes. In addition, multicore optical fiber can support, for example, multiple optical paths in a common cladding with a relatively small diameter (instead of just one optical mode in a cladding in a single-mode fiber), thereby allowing more complex optical fields to be measured and/or created at the distal end of a small, flexible, and low-cost endoscope.

There are numerous medical and non-medical applications of sensor or imaging endoscopes including cardiology, gastroenterology, pulmonology, laparoscopy, sensors, and non-destructive evaluation and non-destructive evaluation and test (NDE/NDT) applications. There are many types of rigid and fixed endoscopes such as classic endoscopes, catheters, imaging guidewires, laparoscopes, borescopes, imaging needles, and other approaches used to relay information from a sensor or from a distal location to a proximal location. Also, there are many approaches to transferring imaging information through a fiber-based instrument including, for example, utilizing single mode or multimode fibers, fiber optical bundles, mechanical or electrooptical scanning elements, sets of relay lens, and graded index lenses.

Recently, the use of multicore optical fibers, for example uses in systems with three or more cores, in endoscopic applications has been described. One of the challenges of using multicore optical fibers with three or more cores in fiber-based instruments is the implementation of the distal optics. Some example requirements of the distal optics for many state-of-the-art applications include that the distal optics be small, precisely positioned with respect to the cores, exhibit high optical quality, inexpensive, exhibit low loss, exhibit low-crosstalk, and/or exhibit low-back reflection. In particular, in many current applications, the distal optics needs to fit on the end of a tiny multicore optical fiber.

The present teaching describes approaches to the implementation of multicore fiber measurement systems that utilize 3D printed optics as part of the distal optics. Although, the term “endoscope” is used in this description, it should be understood the apparatus and method of the present teaching is applicable to a wide range of medical and non-medical instruments such as, but not limited to, those mentioned above. In general, the present teaching describes a system and method for transferring light to and/or from the end of a multicore fiber that is compact, flexible, and easily adapted to multiple illumination and collection configurations, and that exhibits numerous other beneficial features through the use of 3D printed distal optics.

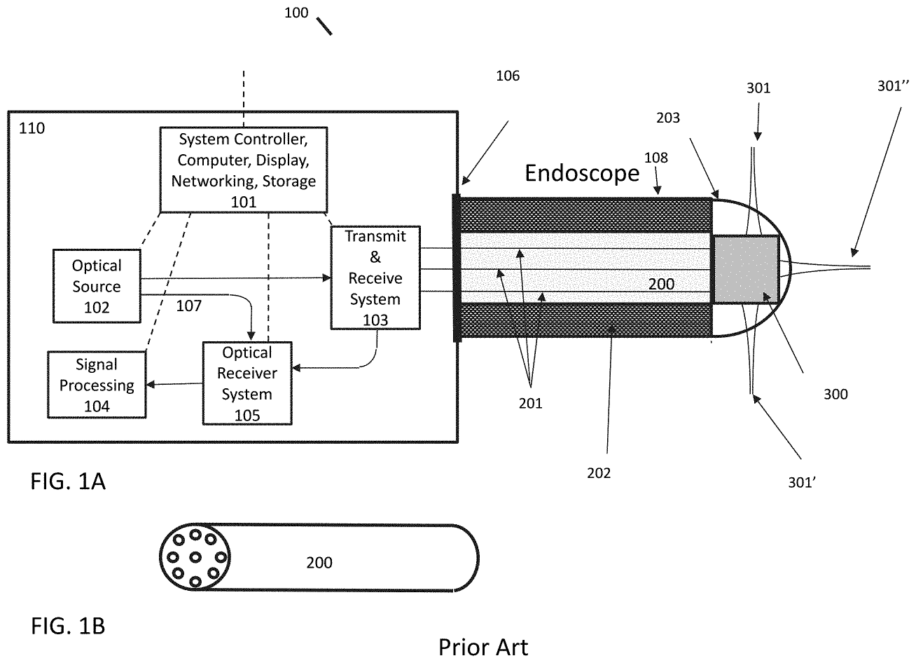

FIG. 1A illustrates a known example general system concept of an endoscopic instrument 100 using a multicore optical fiber 200 with at least three optical cores 201. The endoscopic instrument 100 may be part of, for example, a ranging, sensor and/or imaging system. A console 110 can be configured to implement any one or a number of, for example, Optical Coherence Tomography (OCT), Raman, Fluorescence, or other types of optical or electrooptical sensor or ranging, sensing, or imaging approaches. The console 110, for example, may include an optical source 102, a transmit and receive system 103 that couples the console to the endoscope 108, an optical receiver system 105, a signal processing system 104, and other proximal-end subsystems 101 that may include a system controller, computer, display, networking interface, and/or storage in communication with the other elements 102, 103, 104, and 105 as well as communication with external systems or human interfaces (illustrated by dashed lines). The optical source 102 may be one or more optical sources. For example, the optical source 102 could be a single frequency laser, frequency swept laser, or a broad bandwidth optical source. For some consoles 110 there may be an optical path 107 from the optical source 102 to the receiver 105. For example, in optical coherence tomography systems, interferometric detection is used and light from optical path 107 is interfered with light from the transmit and receive system 103 in the optical receiver system 105. Optical and mechanical and electronic devices, such as optical circulators, optical switches, filters, reference arms, as may be appropriate for the particular imaging technology, also may be used within the console 110 as known to those skilled in the art.

FIG. 1B illustrates an end-view cross-section of the multicore optical fiber 200 of FIG. 1A. As shown in the example of FIG. 1A-B, the embodiment of endoscope 108 contains a multicore optical fiber 200 with nine cores 201 (three are shown in cross section of FIG. 1A). Multicore fiber 200 of the present teaching uses at least three cores 201. Various embodiments of multicore optical fiber 200 utilize a variety of the many types and geometries of multicore fibers including, for example, single mode cores that do not have tight optical coupling, multimode cores, or a combination of single mode and multimode cores, and/or cores within cores, as well as other configurations.

Practical endoscopes typically include significant structural elements that are not illustrated in FIG. 1A, such as protective covers, protective and structural jackets, proximal connectors, torque cables, and many other active or passive structures. Such structure is shown schematically as structure 202 in FIG. 1A. In many embodiments, there is a proximal end connector 106 that connects the console 110 to the endoscope 108. The endoscope 108 may contain a smooth distal cover shown schematically in 203 near the distal tip, that could include transparent windows to allow light beams 301, 301′, 301″ to pass to and/or from the endoscope 108 to the target or sample (not shown). For simplicity, this disclosure features the distal optics 300 that are used to generate the light beams 301, 301′, 301″ but it should be understood that these other structures are generally part of a practical endoscope instrument 100. The configuration of light beams 301, 301′ and 301″ shown in FIG. 1A is just one example. Persons skilled in the art will appreciate that a variety of patterns of emanating and collected light beams are anticipated to be practiced by the apparatus and methods of the present teaching.

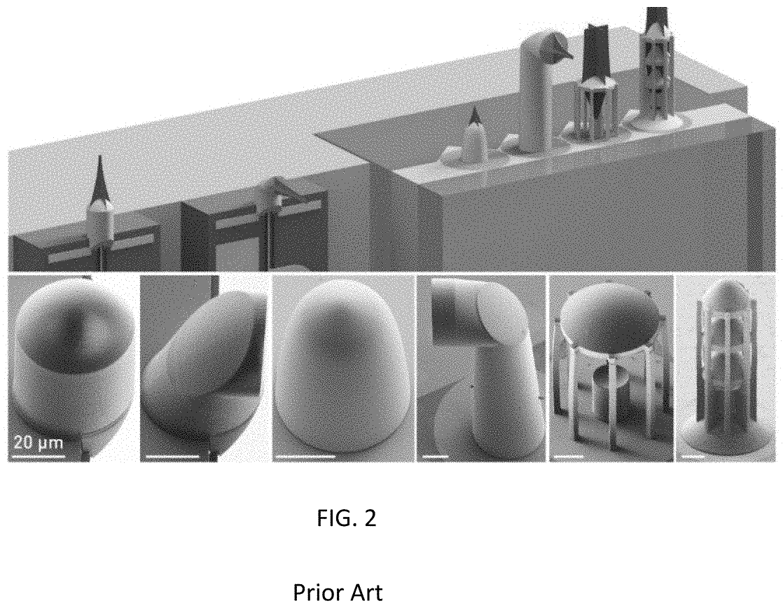

The present teaching describes the use of three-dimensional (3D) printed optics as part or all of the distal optics 300. Three-dimensional printed optics are known in the prior art. FIG. 2 illustrates an example of known 3D printed optics that can be realized. See https://www.vanguard-automation.com/. While various 3D printed optics are known, the application of such 3D printed optics in a multicore fiber endoscope instrument has not been taught. One feature of the present teaching is the use of 3D optics for the implementation of part or all of distal optics of an endoscope with at least three cores in a multicore fiber.

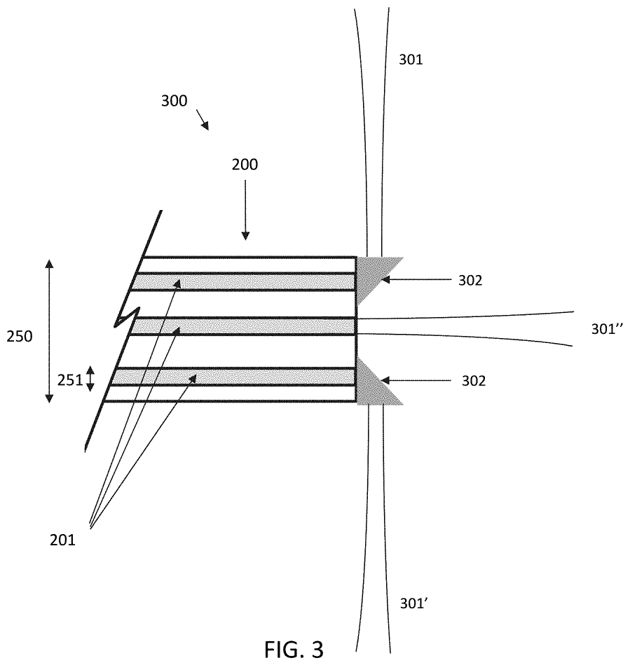

FIG. 3 illustrates an embodiment of a multicore fiber light transfer system 300 in side-view cross section with 3D-printed distal optics fold mirrors 302 of the present teaching. In this embodiment, 3D printing is used to place fold mirrors 302 directly on the end of the multicore fiber light transfer system 300. The multicore fiber light transfer system 300 can be used in a variety of fiber-based instruments to deliver light from a distal end of a multicore fiber 200 and/or to collect light at the distal end of a multicore fiber 200. The multicore fiber 200 transmits the delivered and/or collected light to and/or from the proximal end as appropriate. It should be understood that the additional structure associated with the fiber light transfer system 300, including for example, additional structure 202 and end caps 203 that are described in connection with FIG. 1A, or otherwise associated with fiber-based systems that transfer light, may also be included, but are not shown for simplicity. In the embodiment illustrated in FIG. 3, the fold mirrors 302 are placed on the outer cores of the fiber 200, and the center core does not have a fold mirror. Various embodiments can place fold mirrors 302 on various cores 201, depending on the applications. In the embodiment shown in FIG. 3, the optical beams 301, 301′ on the outer edge of the multicore fiber are directed at 90-degrees from the end face of the fiber 200. Optical beam 301″, at the center of the fiber light transfer system 300 with no fold mirror projects in a forward direction from the end face of the fiber 200. Fold mirror 302 may also be referred to as a beam director or beam deflector. Various embodiments of the fold mirror 302 can operate via reflection or refraction of combinations thereof. For example fold mirror 302 could work as a transmission prism and allow more forward directed imaging. In general fold mirrors (or beam director elements) 302 can be used to allow both radial and forward imaging and sensing. That is exit beams 301 and 301′ do not need to angle at 90 degrees from the fiber axis and the exit angles need not be the same. Exiting at a non-90-degree angle can be beneficial including exiting at deflection angles where total internal reflection or Brewster's angle can be utilized. In addition, or instead, of using total internal reflection, the surface of the fold mirror 302 can be metal or dielectric coated to enhance performance. To minimize back reflection at the fiber-to-fold-mirror interface, the fiber can be cleaved at an angle and that angle can be compensated in the 3D printed fold mirror. This also applies to the exit surface of the fold mirror and any other surfaces where unwanted back reflections could occur. This embodiment is not shown in FIG. 3.

Although no lenses are shown in the configuration illustrated in FIG. 3, it is possible to add one or more lenses, either before or after the fold mirror or both, as described in more detail below. The fold mirror 302 can use reflection or refraction. The surface of the fold mirror 302 not in contact with the fiber 200 could be air, gas, or it could be metal or dielectric coated for enhanced performance as mentioned above. Additional material to secure the 3D printed material that comprises the fold mirror 302, such as UV or other glue, can be added to ensure ruggedness of the attachment of the mirror 302 to the fiber 200. The coating and/or adhesive and/or other additional material can be applied during the 3D printed or added after printing. Referring also to FIG. 1A, a protective cover 203 and other structure 202 (e.g. fiber buffer material, etc.) are not shown for simplicity. Typical outside diameters 250, of the multicore fiber can be 80 to 250 micrometers. Typical core diameters 251, can be 5 to 50 micrometers. In one particular embodiment, the outside diameter 250 is ˜125 micro meters and the core diameter 251 is ˜9 micrometers. Also in some embodiments, it not necessary that all the cores have the same diameter. In some embodiments, the cores overlap. In some embodiments, the cores do not overlap.

In another important embodiment of the present teaching is that the reflecting (or refracting) surface of one or more of the fold mirrors 302, (e.g. the surface between fiber interface and the exit aperture) can be non-planar. Such a surface can create focusing or more generally implement various optical properties such as spherical, aspherical, cylindrical, extended-depth-of focus, or other desirable optical properties on the beams 301 and 301′. Importantly this can allow the fold mirrors 302 to act both as a beam director or beam deflector and a focusing lens in a very compact volume with minimal back reflection, costs, and scattering. The geometry of the fold mirror surface could also compensate for any cylindrical or other aberration introduced along the beam path from the fiber facet to or from the sample (such as those caused by the structures in 203 or 202). Similarly, it is possible to add a 3D printed structure such as a lens to the exit of the center channel to add a beam expander and/or lens property to beam 301″ (not shown in FIG. 3).

Referring to FIG. 3, it should be understood that the fold mirror 302 does not need to be placed immediately on top of the fiber facet where it quickly begins to alter the direction of beam propagation. It is possible to elongate the structure to allow the beam to diverge a bit before its direction is altered by the fold mirror 302. A larger beam diameter combined with focusing surface allows for longer focal lengths and longer depth of fields and instead of the beam immediately diverging, as illustrated in FIG. 3, beams 301 and 301′ can be focusing or converging. An elongation region can be 3D printed or could, for example, be a section of coreless fiber.

It is also possible that there could be more than three multicore fibers and/or no central core and/or each of the beam angles (e.g. beam 301) are slightly different.

One feature of the 3D printed distal optics of the present teaching is that the elements can be printed with a small feature size that is well matched to the multicore fiber size. For example, one printed element can be directly aligned to one core and a second element to a second core. In some cases, the distance between the optical cores is less than 100 microns. In some embodiments, the elements can be printed so that they are less than 100 microns apart. In general, the printed optical elements can be arranged and spaced to match the core pattern of a multicore fiber. Additional material can be added along all or part of the circumference of the facet of the multicore fiber so as to increase structural integrity. During manufacturing, the 3D printing can be aligned to the multicore fiber by a combination of one or more known techniques, such as imaging the side of the fiber from one or more angles (such as used in multicore fiber fusion splicers), imaging the end face of the fiber, using various types of illumination including transmission and reflection of white light, and/or actively coupling light into the individual cores of the multicore fiber at the proximal end.

One feature of the 3D printed fold mirror 302 of the present teaching is that the 3D printing process allows for careful placement of the fold mirror 302 with respect to the fiber 200 end face. Particular desired alignments can be achieved, and these desired alignments can be different for different cores 201 in the fiber 200. In addition, different orientations of the fold mirrors 302 can be provided for different applications and/or for particular cores 201. For example, different angles, focusing properties, and different beam diameters and other optical features may be provided. As a result, a variety of desired patterns and or directions for the optical beams 301, 301′, 301″ can be provided. This kind of flexibility is difficult to achieve using bulk-optic elements for the distal optics because of the complexity and cost to implement. An additional feature of the 3D printed distal optics is that it is easy to achieve precise alignment of the optical elements to the fiber cores.

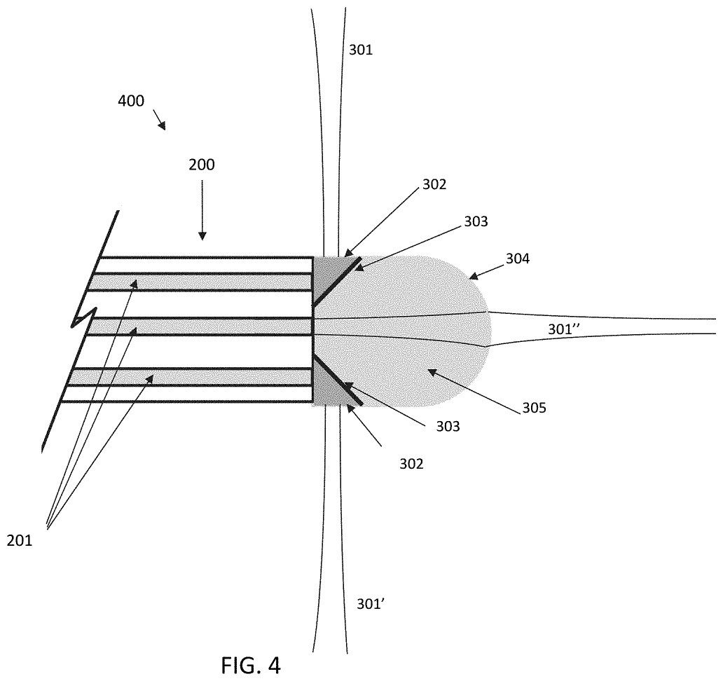

One feature of the apparatus of the present teaching is that it is possible to provide additional structure during or after 3D printing to provide additional capabilities. For example, it is possible to fill in the space formed by the fold mirrors 302. FIG. 4 illustrates an embodiment of a multicore fiber light transfer system 400 in side-view cross section with 3D-printed distal optics fold mirrors 302 of the present teaching with additional distal structure. In this embodiment, an enhanced reflection coating (or air space or dielectric or metal material coating) 303 is formed over the fold mirrors 302. As mentioned above this is also a surface coating 303 that can be put on a non-planar surface and introduce desirable optical properties such as focusing, aberration correction, or extended depth of field. In some embodiments, a structure 305 is formed by 3D printing that fills the area at the output of the end face of the fiber 200. The structure 305 has a curved surface 304 that forms a lens, thereby focusing optical beam 301″ that passes in the forward direction from the end face. The interior of the structure 305 may be solid, as indicated, or there may be internal structure that includes, for example, additional air-gap regions or regions with different refractive index or a metal coating between mirrors 302 and lens surface 304. One feature of 3D printing optics is the ability to produce air-gaps of a variety of shapes internal to an optical element.

In some embodiments, different refractive index materials are used to create the center lens surface 304 and appropriate outer surface coating 303 on fold mirrors 302. Although 90-degree beam projections and right angles are shown in the various diagrams of this disclosure, it is understood that those angles can be altered to minimize back reflections and/or get different imaging configurations and/or to optionally harness total internal reflection of the light from the cores 201 to the external beams, 301, 301′, 301″, that imping on the sample (not shown) whose optical properties, or other parameters (e.g. distance, range, or chemical composition), are to be measured. In one embodiment of the configuration shown in FIG. 4, the side beam directed beam paths 301, 301′ do not have lens elements but the forward directed beam 301″ does have lens elements. In another embodiment the surfaces of the fold mirrors 302 are not planar and can have lensing properties and the lens elements themselves can have some beam expansion region between the exit facet of the fiber 200 and the deflection of the fold mirror 302. This can be beneficial when the cores 201 of the multicore fiber 200 are single mode cores as such beams can diverge quite quickly due to the small exit aperture and the laws of optical beam propagation.

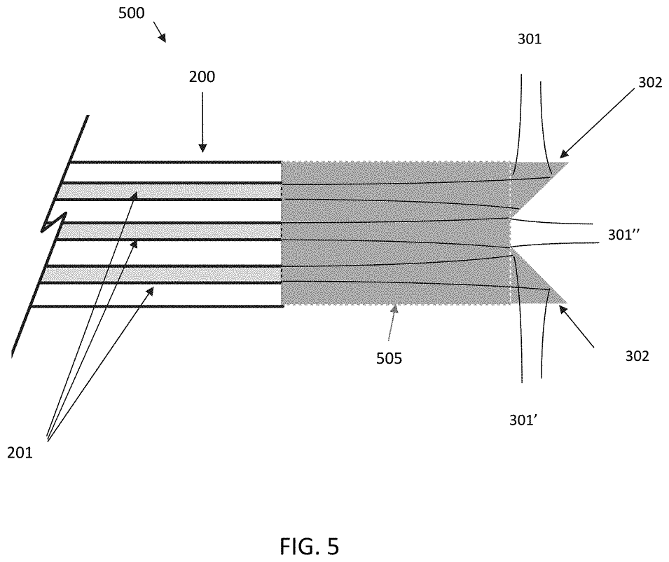

FIG. 5 illustrates an embodiment of a multicore fiber light transfer system 500 in side-view cross section with distal optics comprising a lens 301 before fold mirrors 302 of the present teaching. In some embodiments, the lens 301 is a bulk lens or a fiber lens. In some embodiments the lens 301 is a 3D printed lens. A multicore fiber light transfer system 500 includes distal optics that includes an optic 505 that is attached to the end face of the fiber 200 and positioned before end mirrors 302. The optic 505 can impart one or more optical properties on beams 301, 301′ and 301″.

The optic 505 can be realized with many types of lenses. For example, in some embodiments, the optic 505 is a bulk optical GRIN lens. In other embodiments, the optic 505 is a fiber GRIN lens, like a single or multicore fiber GRIN lens. Such a fiber lens can be fusion spliced to the fiber 200 or attached by other means. But in some embodiments, the multicore GRIN lens is not a fiber GRIN lens. The optic 505 can be a single device or multiple devices. Also, in some embodiments, the optic 505 is 3D printed. In addition, optic 505 could have both a beam expansion region to allow the light to or from the fiber 200 to grow in diameter followed by lensing properties such as focusing. In addition to imparting optical properties of the light to and/or from fiber cores 201 using optic 505 the one or more of the surfaces of fold mirrors/beam directors elements 302 can be non-planar to impart additional desired optical properties on beams 301, 301′, and 301″ such as longer focal lengths, extended depth-of-field, and/or cylindrical aberration correction.

In some embodiments, fold mirrors 302 are attached to the optic 505. The fold mirrors 302 in various embodiments may operate in reflection and/or refraction. In some embodiments, the fold mirrors 302 are 3D printed directly on the end of the optic 505. The center channel optical beam 301″ is optional as there can be some advantages to not having a center channel. For example, not having a center channel can allow the beams that emerge from the end face of the fiber 200 to diverge more before they interfere with one another. In this way, a bigger diameter beam exiting the light transfer system 500 can be supported and hence a different numerical aperture is used to further optimize focal spot location, diameter, and depth of field.

One feature of the methods and apparatus of the present teaching is that it is possible to fill in the area forwards of the tip of the apparatus shown with material and structure that allow a smooth outer surface, enhanced transmission, ensure structural integrity, or even to provide a radio-opaque tip to, for example, show up on a x-ray or ultrasound image.

In another embodiment (not shown), there are no fold mirrors 302 in FIG. 5, and instead the optic 505 includes multiple lens and/or other optical elements, each element centered on a core 201. This embodiment of optic 505 could be simply 3D printed on the end of the multicore fiber 200 and all beams 301, 301′, 301″ are forward directed imaging. The beams could optionally focus forward of the end of the distal optic in the same location or different locations in the lateral plane (1D or 2D) or the longitudinal plane or both. For example, all three beams 301, 301′, 301″ could focus parallel to one another and at the same distance from the end of the fiber but at a different location. This can be achieved by a single lens or multiple lenslets in optic 505. Alternatively, the beams can all be forward focusing but at different angles (e.g. a conical pattern with a center beam) so that the lateral footprint of the focal spots is much wider than the diameter of the fiber 200. Such a beam pattern can be important in for example optical coherence tomography systems in otolaryngology inner ear applications where multiple measurements of inner ear properties are needed and mainly forward imaging is needed but cost and size do not permit the use of mechanical scanning mechanism to scan a single beam.

In another embodiment (not shown), the individual fold mirrors/beam directors 302 are positioned prior to the optic 505, which is positioned between the multicore fiber 200 and the optic 505. This configuration can, for example, allow all or some of the beams 301 and 301′ to focus near the same spot along the optical axis. This configuration can also be used to allow all the beams 301 and 301′ and 301″ to focus at different spots along the optical axis. In another embodiment, the individual fold mirrors/beam directors 302 are positioned after the optic 505, which is positioned between the multicore fiber 200 and the optic 505. As indicated, the optic 505 can include multiple lenslet surfaces or a single lens surface. Also, the multiple lenslet surfaces can be aligned to particular cores of the fiber light transfer system 200.

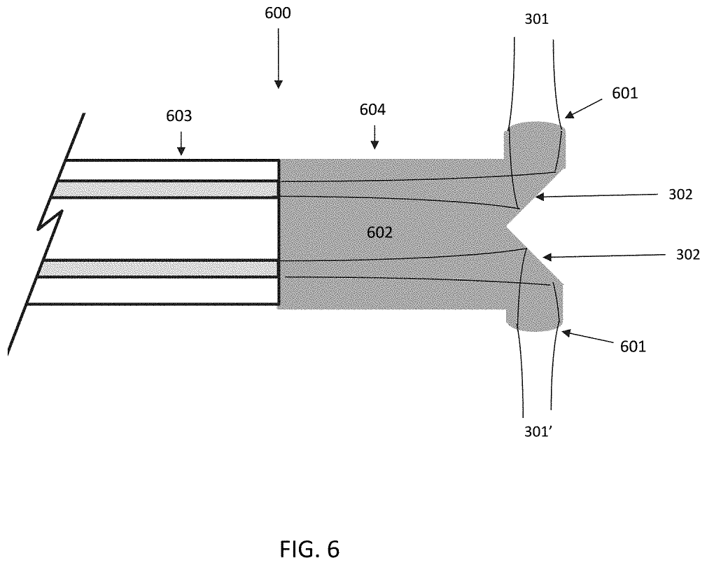

FIG. 6 illustrates an embodiment of a multicore fiber light transfer system 600 in side-view cross section with multicore fiber 603 and with distal optics including 3D printed fold mirrors 302 positioned forward of 3D printed lenses 601 of the present teaching. The distal optic element 604 can have an optional beam expansion region 602 positioned between the end face of the fiber 603 and the individual fold mirrors 302. The entire structure could be 3D printed, or only some parts of the structure are 3D printed. Individual lenses 601 are positioned after the fold mirrors 302. As mention above, the surface of the reflecting surface of fold mirror 302 could be configured to reflect the beams 301, 301′ at angles other than 90 degrees. The fold mirror 302 can work on total internal reflection or be HR coated, and/or the surface could be non-planar to introduce additional optical beam shaping and optical propagation properties.

As mentioned above, the multicore-fiber-to-3D-printed-distal-optic interface to the end face of fiber 603 can be angled to minimize back reflection (not shown). In another embodiment (not shown) an additional center optical beam is provided by a central core in fiber 603 to support forward sensing or imaging. This can be done by shorting the beam expansion region 602 and reducing the relative diameter of each fold mirror 302.

The various embodiments of multicore fiber and 3D printed distal optics described in connection with FIGS. 3-6 are a significant improvement over prior art multicore fiber and distal optics of known systems, such as that illustrated in FIGS. 1A-B. In some configurations the ability to do 3D printing allows unique distal optics to be practically manufactured for the first time. For example, the distal optics 300 and/or protective cover 203 illustrated in FIG. 1A can be practically replaced by the distal optics configurations of FIGS. 3-6 as well as combinations of all or part of these distal optics configurations.

It should also be noted that it is possible to add mechanical or electro-optical scanning mechanisms to the various embodiments shown in FIGS. 1, 3-6. For example, the entire endoscope could be placed in a reciprocating rotational configuration with or without a pullback mechanism as in known art to perform a level of circumferential scanning with pullback scanning of a lumen in for example an endoscopic optical coherence tomography system embodiment. This could allow for much more rapid scanning since multiple A-line scans could be acquired in parallel. In one embodiment suitable for intraluminal imaging, there are multiple beams (e.g. 301 and 301′ in various figures) that exit at different angles and the fiber is rotated back and forth nearly 360 degrees and the multiple beams sweep out a larger area of the lumen than would be achieved in a single core fiber and thus allow more rapid measurements of the optical properties of the lumen. Also, scanning could just include longitudinal pullback with no automation in rotation. It is also possible to add distal motors with spinning or beam deflecting mirrors and keep the fiber stationary.

There are also embodiments in otolaryngology where a very low-cost probe is needed that produces multiple beams of the inner ear topology. Conventional approaches using scanning elements tend to be larger and more costly than an embodiment using a multicore optical fiber with 3D printed distal optics that can perform many axial measurements at once.

There are also embodiments in imaging or sensing guidewire applications. Guidewires need to be very small and flexible but one of the challenges of guidewires is how to control and navigate within a torturous channel of arteries and veins. This includes when trying to pass blockages such as in CTO crossings where it can be unclear where to navigate and what is artery wall and what is blocked artery. Using conventional rotational spinning and forward imaging OCT approaches can result in too big a guidewire due to the need for having large torque cables or other structure. By using a multicore fiber with 3D printed optics, it is possible to have multiple forward directed A-Scans in an OCT imaging application to help guide the guidewire to properly navigate the arterial channel.

A further embodiment relates to intraluminal and other medical applications including guidewire, endoscopes, catheters, robotic surgery, and other medical devices used within the human body where in addition to sensing, measuring, and/or imaging structural information, functional information is important. Functional information includes, for example, the qualitative or quantitative measurement of motion or flow. As one example, the flow could be from a liquid, such as blood or a saline flush flowing inside a human artery or vein. There are many other sources of motion from moving gases, liquids, beating hearts, ciliary motion, and more. Motion can be measured in many ways including speckle decorrelation and/or Doppler techniques as is known in field of OCT, laser vibrometery, and other biomedical imaging modalities.

As illustrated herein, there are many geometries that are possible with the multicore fiber including multiple beams aimed at an angle less than 90 degrees (back reflected), an angle of 90 degrees (right angle), an angle more than 90 degrees (forward imaging), and/or combinations of all of these. By incorporating functional information (e.g. flow) in a multicore fiber endoscope with 3D printed lenses, it is possible to create a small, flexible, and inexpensive endoscope that can yield both structural and functional information and that can provide additional diagnostic information (e.g. virtual fractional flow reserve (FFR)) or guidance information (e.g. which way to steer a guidewire to remain in the lumen flow and not puncture the artery wall). As one example, if the multicore fiber has a forward imaging configuration and the artery is sharply curving one way, then flow differential from the various forward imaging fiber cores will yield information about that curve that can be used for guidance and/or diagnostic information. In general, by looking at ratios of structural and/or functional information from the various channels of the multicore fiber and using 3D printed lenses, a small flexible low-cost endoscope configuration is possible without the requirement of having a continuously rotating endoscope as is commonly used in today's intravascular OCT products or a complex mechanical forward scanning imager.

In some embodiments, illumination from a sample or a target is passed through a 3D printed element such that structural and/or functional information from the sample or the target is coupled into the plurality of cores of a multicore fiber, and where the light in each core is considered as a separate an information channel. The light from each information channel is received at a proximal end of the multicore fiber and is processed to provide guidance or diagnostic information about the sample or the target. For example, phase and/or amplitude and/or spectral information can be determined about the light in each channel using the proximal receiver, and then this information is subsequently processed.

One skilled in the art will appreciate that there is that a wide variety of possible configurations for multicore fiber with the distal optics that can be realized using the 3D printing according to the present teaching. For example, the distal optics can contain beam expansion regions, fold mirrors, beam directors, lenses and a variety of materials some of which can be 3D printed directly on the end of a multicore fiber with very precise positioning. These elements can also be 3D printed directly on a lens element that is attached to the multicore fiber. Furthermore, these elements can be 3D printed directly on another material that is attached to the multicore fiber.

Another feature of the apparatus of the present teaching fabricated with 3D printing is that a variety of exit angles for the optical beams that emerge from the different cores in the multicore fiber can be easily achieved. This includes, for example, backward imaging angles, side imaging angles, and forward directed imaging angles. In various configurations, the projection for various types of fold mirrors (or beam directors) can be achieve through reflection, refraction, absorption, scattering or numerous combinations thereof. A variety of known beam directing techniques can be implemented with the 3D printing approach.

An advantage of the 3D printing described herein is that multiple different kinds of beam directing elements can be included in a single distal optical element. Another advantage is lower cost for higher complexity of optical structures as compared to bulk-optic or fiber-based solutions. Various embodiments described herein clearly illustrate how a single 3D printed element can replace multiple bulk or fiber-based elements of known fiber-based instruments, which reduces back-reflections and reduces cost and complexity and can achieve superior optical quality.

One feature of the present teaching is that it provides a method for manufacturing distal optics for a multicore optical fiber for a fiber-based instrument. The method includes the first step of preparing and fixturing an end face of an optical fiber with multiple cores for printing. This may include, for example, providing an angled facet to reduce reflections. In a second step, an optical material is printed onto the end face of the fiber. It is possible to have the fixturing to allow for multiple fibers to be placed in the 3D printer to reduce setup on time and increase manufacturing throughput. The material may be 3D printed into a shape that may include at least one planar and/or one curved surface that is in the optical path of at least one of the cores in the multicore fiber. This shape can provide alteration of an optical beam that emerges to or from the core aligned to the shape. The shape may be configured, e.g. as illustrated by element 304, 505, 601 in FIGS. 3-6.

The material can be 3D printed into a shape that can also or instead include at least one flat or non-flat surface with an angle that is different from the angle of the end face of the fiber, and that is aligned to at least one of the cores in the multicore fiber. This angled flat or non-flat surface provides for directing an optical beam that emerges from the core aligned to the angled flat or non-flat surface in a desired direction that is set by the optical properties of the flat or non-flat surface. The angled flat or non-flat surface may be configured, e.g. as illustrated by element 302 in FIGS. 3-6.

The material can also be 3D printed into a shape that includes a uniform propagation region with a particular length along the beam path. This uniform propagation region can be aligned to at least one of the cores in the multicore fiber or the entire multicore fiber itself (or any subset). This uniform propagation region provides for beam expansion of an optical beam that emerges from the core aligned to the uniform propagation region wherein a desired beam expansion is achieved by a chosen uniform propagation length.

The materials that are printed into these various shapes can be the same material or a different material can be used for one or more of the different shapes. One or more of the various shapes can be printed in different positions along a direction of an optical beam path. The particular shapes and their relative positions printed along a path for two different beams can be the same or different.

In an optional third step of the method, a bulk-optic and/or fiber-based optical element is attached to the end face of the multicore fiber. In some embodiments, this step is performed before the 3D printing step. In some embodiments, a bulk-optic and/or fiber-based optical element is attached to the 3D printed element after it is printed on the end face of the fiber. As is understood by those skilled in the art, the steps of the method can be applied in various orders as appropriate to the desired configuration of the distal optical elements attached to the multicore fiber in the fiber-based instrument of the present teaching. Thus, it is possible to have both 3D printed and non-3D printed materials in the distal optic 300. One example is a section of coreless fiber spliced to the multicore fiber, followed by a section of multimode GRIN fiber fusion spliced to the coreless fiber, and then, fold mirrors or other 3D printed structures on the distal surface.

In an optional fourth step, additional material is added to partially or wholly cover the 3D printed elements. Examples are an UV glue, or other adhesive to help secure the 3D elements.

The steps of the method for manufacturing distal optics for a multicore optical fiber for a fiber-based instrument described herein result in a multicore fiber instrument with fully or partially 3D printed distal optics that are able to project and/or focus one or multiple optical beams to a target surface with a desired direction and beam shape that optimizes a particular illumination and/or measurement as described herein. In some embodiments, the 3D printed distal optics is able to collect illumination from a target surface and transmit that collected illumination through the multiple cores of the fiber to a receiver at a proximal end. The 3D printed distal optics that result from the steps of the method for manufacturing distal optics for a multicore optical fiber for a fiber-based instrument have many advantages over the prior art such as they can be smaller, exhibit higher optical quality, be manufactured at lower cost, be lower loss, exhibit lower crosstalk, and/or exhibit lower back reflection compared with prior art distal optics manufactured with only bulk-optic or fiber optic elements. The 3D printed optics provides more flexibility in the number and types of optical elements that can be constructed. In addition, there is a wider variety in the resulting beam patterns that can be achieved from the output of multicore optical fibers with the 3D printed optics.

Referring to FIGS. 1A and 3-6, in some configurations according to the present teaching, light emanating from at least some fiber cores 201 is directed and/or focused and/or otherwise modified by a 3D printed optical element and/or a bulk-optic or fiber optic element such that the emanated light illuminates a target, sample, bodily tissue or other element to be measured with a desired optical beam pattern. In some embodiments, light is collected from a target, sample, bodily tissue or other element to be measured using a 3D printed optical element and/or a bulk-optic or fiber optic element described herein and passed to at least some fiber cores 201 so that the collected light can be passed to a receiver 105 in a console 110 such that the fiber-based measurement system provides information about the optical properties of the target, sample, bodily tissue or other element to be measured.

In some embodiments, an optical beam from one core passes through a 3D printed lens shape that focuses the beam at a desired location along a path in the forward direction and an optical beam from another core passes through a 3D printed fold mirror shape that directs the optical beam on a path that is at an angle with respect to the forward direction. In some embodiments, the 3D printed lens shape and the 3D printed fold mirror shape are printed as a single continuous structure. Also, in some embodiments, the 3D printed lens shape and the 3D printed fold mirror shape are printed from a single material. In other embodiments, the 3D printed lens shape and the 3D printed fold mirror shape are printed from different materials. In other embodiments a 3D printed optic is in optical contact with materials that are not 3D printed but applied after 3D printing (e.g. metal or dielectric coatings, UV epoxies, etc.)

### EQUIVALENTS

While the Applicant's teaching is described in conjunction with various embodiments, it is not intended that the Applicant's teaching be limited to such embodiments. On the contrary, the Applicant's teaching encompasses various alternatives, modifications, and equivalents, as will be appreciated by those of skill in the art, which may be made therein without departing from the spirit and scope of the teaching.

What is claimed is:

1. A multicore fiber light transfer system comprising:
a) a multicore fiber having a proximal end and a distal end and at least three optical cores, the multicore fiber transferring light from the proximal end to the distal end and collecting light from a target at the distal end and transferring the collected light to the proximal end; and
b) distal optics that is 3D printed near the distal end of the multicore fiber, the distal optics comprising a first element having a surface that is aligned to one core of the multicore fiber with a first shape such that the first element projects the light transferred from the proximal end in a first desired direction with a first desired beam shape and having a second element comprising a surface that is aligned to another core of the multicore fiber with a second shape such that the second element collects light from a desired location on the target.

2. The multicore fiber light transfer system of claim 1 wherein the first element and the second element are 3D printed using the same material.

3. The multicore fiber light transfer system of claim 1 wherein the first element and the second element are 3D printed using two different materials.

4. The multicore fiber light transfer system of claim 1 further comprising a bulk optic element positioned between the multicore fiber and at least one of the first and second element.

5. The multicore fiber light transfer system of claim 1 further comprising an adhesive material that at least partially covers the distal optics.

6. The multicore fiber instrument of claim 1 wherein the second element further comprises a coating that at least partially covers the flat surface.

7. The multicore fiber light transfer system of claim 1 wherein the distal end face of the multicore fiber is oriented perpendicular to the central axis of the multicore fiber.

8. The multicore fiber light transfer system of claim 1 wherein the distal end face of the multicore fiber is oriented off a perpendicular to the central axis of the multicore fiber.

9. The multicore fiber light transfer system of claim 1 wherein a distance between the position of the first element and the second element is less than 100 microns.

10. The multicore fiber light transfer system of claim 1 wherein the first element comprises a curved surface that is aligned to one core of the multicore fiber.

11. The multicore fiber light transfer system of claim 1 wherein the distal optics is 3D printed near the distal end of the multicore fiber.

12. The multicore fiber light transfer system of claim 1 wherein the distal optics is 3D printed on the distal end of the multicore fiber.
